SEQUENCE LISTING (24 sequences)

SEQ ID NO: 1 - DNA, 20 bp, Artificial Sequence
  Primer
cgaccatctgccgctttgag

SEQ ID NO: 2 - DNA, 20 bp, Artificial Sequence
  Primer
ccccctgtcccccattccta

SEQ ID NO: 3 - DNA, 26 bp, Artificial Sequence
  Primer
agcatccgactgtaaagaatcttcac

SEQ ID NO: 4 - DNA, 24 bp, Artificial Sequence
  Primer
cggccagttgtttttctgccacct

SEQ ID NO: 5 - DNA, 18 bp, Artificial Sequence
  Primer
cccccggcggcaatagca

SEQ ID NO: 6 - DNA, 20 bp, Artificial Sequence
  Primer
tcggcgccggggagatacat

SEQ ID NO: 7 - DNA, 19 bp, Artificial Sequence
  Primer
ggagctatgttgtacctcc

SEQ ID NO: 8 - DNA, 19 bp, Artificial Sequence
  Primer
gtgggccatttccagaggg

SEQ ID NO: 9 - DNA, 26 bp, Artificial Sequence
  Primer
agaaagtagtgatactcaaggaccaa

SEQ ID NO: 10 - DNA, 26 bp, Artificial Sequence
  Primer
tgacagagattagcttcttcaaaagt

SEQ ID NO: 11 - DNA, 20 bp, Artificial Sequence
  Primer
gtccgtggctacctgtcatt

SEQ ID NO: 12 - DNA, 20 bp, Artificial Sequence
  Primer
tggcacccaactctcataca

SEQ ID NO: 13 - DNA, 22 bp, Artificial Sequence
  Primer
gtgaaactctgggagattctcc

SEQ ID NO: 14 - DNA, 20 bp, Artificial Sequence
  Primer
cgacattcaattgccatgag

SEQ ID NO: 15 - DNA, 21 bp, Artificial Sequence
  Primer
atgagagccctcacactcctc

SEQ ID NO: 16 - DNA, 21 bp, Artificial Sequence
  Primer
gccgtagaagcgccgataggc

SEQ ID NO: 17 - DNA, 25 bp, Artificial Sequence
  Primer
gagaaggtttcagaagactcagtga

SEQ ID NO: 18 - DNA, 24 bp, Artificial Sequence
  Primer
ctagaacagcaaaacacagtagtc

SEQ ID NO: 19 - DNA, 19 bp, Artificial Sequence
  Primer
agcctgcgctccaatgact

SEQ ID NO: 20 - DNA, 20 bp, Artificial Sequence
  Primer
tggaacacgatgcctttcac

SEQ ID NO: 21 - DNA, 18 bp, Artificial Sequence
  Primer
cctatgatgaagcggtgc

SEQ ID NO: 22 - DNA, 20 bp, Artificial Sequence
  Primer
tatctgggaaacccacgaag

SEQ ID NO: 23 - DNA, 25 bp, Artificial Sequence
  Primer
tgaaggtcggagtcaacggatttgg

SEQ ID NO: 24 - DNA, 24 bp, Artificial Sequence
  Primer
catgtgggccatgaggtccaccac

(19) United States
(12) Patent Application Publication
(10) Pub. No.: US 2011/0008764 A1
(43) Pub. Date: Jan. 13, 2011

(54) HUMAN GONADAL STEM CELLS

(75) Inventors: Francisco J. Silva, Tustin, CA (US); Rafael Gonzalez, Placentia, CA (US)

Correspondence Address: FISH & RICHARDSON P.C. (NY), P.O. BOX 1022, MINNEAPOLIS, MN, 55440-1022, US

(73) Assignee: DAVINCI BIOSCIENCES LLC, Costa Mesa, CA (US)

(21) Appl. No.: 12/792,202

(22) Filed: Jun. 2, 2010

Related U.S. Application Data
(60) Provisional application No. 61/183,173, filed on Jun. 2, 2009.

(51) Int. Cl. A01N 1/02; C12N 5/074

(52) U.S. Cl. 435/2; 435/366

(57) ABSTRACT

Adult human gonadal stem cells that are capable of differentiating into cells of the mesodermal lineage and ectodermal lineage are described.

### CROSS-REFERENCE TO RELATED APPLICATIONS

This application claims the benefit of priority of U.S. Provisional Application Ser. No. 61/183,173, having a filing date of Jun. 2, 2009, the disclosure of which is incorporated herein in its entirety.

### TECHNICAL FIELD

This invention relates to gonadal stem cells from humans, and more particularly, to gonadal stem cells from adult human testes.

### BACKGROUND

Recent data on cell transplantation into animal models of degenerative diseases and injuries illustrate the feasibility of using adult stem cells for regenerative medicine. See, Chopp et al., Neuroreport 11: 3001-5 (2000); Onda et al., J Cereb Blood Flow Metab. 1-12 (2007); and Prockop, Clin Pharmacol Ther. 82(3): 241-3 (2007). Mesenchymal stem cells (MSCs) are one of the most investigated adult stem cells. Success in transplantation of these cells stimulated the search for other cell populations from different tissues. It has been illustrated that cells isolated from umbilical cord blood (Kern et al., Stem Cells 24: 1294-1301 (2006)), placental cord blood (Kögler et al., J Exp Med 200(2):123-35 (2004)), adipose tissue (Zuk et al., Tissue Eng. 7(2): 211-228 (2001)), and dental pulp (Ikeda et al., Differentiation 76: 495-505 (2008)) have similar properties to MSCs, yet also possess unique characteristics.

Several groups have reported that following transplantation of adult MSCs, patients' symptoms improved significantly in various disease states. See, e.g., Horwitz et al., Proc. Natl. Acad. Sci, USA, 99:8932-8937 (2002); Assmus et al., Circ Res 100:1234-1241 (2007); and Le Blanc and Ringde'n, J Intern Med 262:509-525 (2007). Despite the uncertainty around the mechanism of adult stem cells action upon transplantation into the injured site, MSCs are presently the most promising tool for cell-based therapies. Studies have demonstrated that MSCs may be supportive to tissue recovery (e.g., Akiyama et al., J. Neurosci. 22(15):6623-30 (2002)), stimulate the synthesis of cytokines and matrix molecules([Prockop et al., supra), be angiogenic (Onda et al., supra), have immunomodulatory effects (LeBlanc and Ringde'n, supra), and stimulate endogenous tissue progenitors (Prockop et al., supra). Nevertheless, due to the heterogeneity of disease, each disease condition may require different properties from transplanted cells in order to improve the disorder to which the cells are being applied. Thus, there is a need for different cell types for therapeutic applications to address the specific disease condition in the most appropriate way.

### SUMMARY

This invention is based on the discovery of a new population of cells from adult human testis termed gonadal stem cells (GSCs). GSCs are positive for cell surface markers CD44, CD105, CD166, CD73, CD90, and STRO-1 and lack hematopoietic cell surface markers CD34, CD45, and HLA-DR. In addition, GSCs express pluripotent markers Oct4, Nanog, and SSEA-4. GSCs can be propagated for at least 64 population doublings and exhibit clonogenic capability. GSCs also have a broad plasticity and the ability to differentiate into adipogenic, osteogenic, chondrogenic, neurogenic, and cardiogenic cells. The results described herein demonstrate that GSCs can be easily obtained. Therefore, GSCs can be useful for therapeutic applications such as atrophic nonunion, bone fractures, autoimmune diseases, spinal cord injuries, stroke, diabetes, diabetes cardiomyopathy, as well as to repair cartilage and spinal discs (e.g., degenerative disc and meniscus).

In one aspect, this document features a purified population of adult human GSCs, wherein the cells are positive for CD44, CD105, CD166, CD73, CD90, and STRO-1, negative for CD34, CD45, and HLA-DR, and do not express Vasa, Dazl, and Sox2. The cells further can express vimentin, Oct4, and Nanog. The cells also can be further positive for SSEA-4. The cells can be obtained from an adult testis sample. The cells are capable of differentiating into cells of mesodermal lineage (e.g., adipogenic cells, osteogenic cells, chondrogenic, and cardiogenic cells) and cells of the ectodermal lineage (e.g., neurogenic cells). The cells can have undergone at least 40 doublings in culture (e.g., at least 50 doublings in culture or at least 60 doublings in culture). The cells can include an exogenous nucleic acid (e.g., an exogenous nucleic acid encoding a polypeptide). The cells can be housed within a scaffold (e.g., a biodegradable scaffold such as a scaffold composed of collagen).

In another aspect, this document features a clonal line of adult human GSCs, wherein the cells are positive for CD44, CD105, CD166, CD73, and STRO-1, negative for CD34, CD45, CD90, and HLA-DR, and do not express Vasa, Dazl, and Sox2. The cells can be further positive for SSEA-4. The cells are capable of differentiating into cells of mesodermal lineage (e.g., adipogenic cells, osteogenic cells, chondrogenic, and cardiogenic cells) and cells of the ectodermal lineage (e.g., neurogenic cells). The cells can include an exogenous nucleic acid (e.g., an exogenous nucleic acid encoding a polypeptide). The cells can be housed within a scaffold (e.g., a biodegradable scaffold such as a scaffold composed of collagen). The cells can have undergone at least 40 doublings in culture.

In another aspect, this document features a composition that includes a purified population of GSCs or clonal line of GSCs as described above and a culture medium. The composition further can include a cryopreservative.

In yet another aspect, this document features an article of manufacture that includes a purified population of GSCs or clonal line of GSCs as described above. The purified population of cells or the clonal line can be housed within a container (e.g., a vial or a bag). The container further can include a cryopreservative. In some embodiments, the purified population of cells or the clonal line can be housed within a scaffold (e.g., a biodegradable scaffold).

This document also features a method for purifying a population of GSCs from adult human testis. The method includes obtaining cells from a human testis sample, culturing the human testis cells on a fibronectin coated substrate, and purifying the GSCs from the human testis cells by adherence to the fibronectin coated solid substrate, wherein the GSCs are positive for CD44, CD105, CD166, CD73, CD90, and STRO-1, negative for CD34, CD45, and HLA-DR, and do not express Vasa, Dazl, and Sox2. The cells further can express vimentin, Oct4, and Nanog. The cells can be further positive for SSEA-4.

This document also features a method for culturing a population of GSCs from adult human testis. The method includes obtaining a population of GSCs from adult human testis, wherein the GSCs are positive for CD44, CD105, CD166, CD73, CD90, and STRO-1, negative for CD34, CD45, and HLA-DR, and do not express Vasa, Dazl, and Sox2; and culturing the cells in the presence of a growth medium containing glucose, serum, fibroblast growth factor 2, and glial cell derived neurotrophic factor.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A contains a phase contrast image of GSCs (whole population) and a phase contrast image of a GSCs clone (GSC-cs), demonstrating that the GSCs and GSC-cs exhibit differences in morphology when maintained under the same culture conditions (10×). FIG. 1B is a growth curve for GSCs and GSC-cs. FIG. 1C is a graph depicting cumulative population doublings for GSCs and GSC-cs. FIG. 1D is a photograph of the normal karyotype of GSC-cs after seven passages.

FIG. 2 is the flow cytometry analysis of GSCs isolated from testis at passage 3. GSCs express markers indicative of MSCs. Filled histograms—antibody staining, open histograms indicate appropriate isotype controls. Percent of positive cells is indicated for each antigen studied.

FIG. 3A are images of the immunocytochemical analysis of GSCs for pluripotent stem cell markers Oct4, Nanog and SSEA-4. GSCs also express the intermediate filament marker vimentin. Nuclei were stained with DAPI. FIG. 3B is a representative gel of PCR products. Lane 1—whole testes, lane 2—GSCs passage 1, lane 3—GSCs passage 4, lane 4—GSCs passage 9, lane 5—GSC-cs passage 4, lane 6 NT2 control cells.

FIG. 4A contains a photomicrograph of GSCs and a photomicrograph of GSC-cs, where the cells are undergoing adipogenesis (19 days) and the lipid droplets are stained with Oil Red 0 (small inserts are controls). FIG. 4B is a bar graph of the relative expression of lipoprotein liapase and PPARiso 2 at day 12 and day 19 in GSC (filled bars) undergoing adipogenic differentiation. Up regulation was observed in induced GSCs as compared to controls (open bars). FIG. 4C is a graph quantitating dye accumulation/well for Oil Red 0 (ORO) for GSCs and GSC-cs. FIG. 4D contains a photomicrograph of GSCs and a photomicrograph of GSC-cs, where the cells are undergoing osteogenesis (19 days) and calcium deposits are stained with Alizarin Red S (small inserts are controls). FIG. 4E is a bar graph of the relative expression of osteocalcin and DLX5 at day 12 and day 19 in GSCs (filled bars) subjected to osteogenic differentiation. Up regulation was observed in induced GSCs as compared to controls (open bars). FIG. 4F is a graph quantitating dye accumulation/well for Alizarin Red S (ARS) for GSCs and GSC-cs. Increased dye accumulation was observed in induced cells as compared to controls (open bars) for both GSCs and GSC-cs. FIG. 4G contains photomicrographs of GSCs (left panels) and GSCs (right panels) undergoing chondrogenesis (28 days) and stained with Alcian blue for sulfated proteoglycans. Controls are top panels and induced are lower panels. FIG. 4H is a bar graph of the relative expression of aggrecan and link in samples subjected to chondrogenic differentiation. In FIGS. 4A, D, G, photomicrographs are 40×; inserts in G are 4× low magnification. Data are mean +SEM of triplicate samples. The ratio was calculated against the values in control that was set to 1. *, p<0.05; **, p<0.01; ***. P<0.001.

FIG. 5A is an image from the immunocytochemical analysis of differentiated GSCs for cardiac markers Desmin and Troponin T (40×). FIG. 5B is an image from the immunocytochemical analysis of differentiated GSCs for the neural marker Nestin (10×). Nuclei are stained with DAPI.

### DETAILED DESCRIPTION

In general, this document provides purified populations of gonadal stem cells (GSCs) from adult human testes and clonal GSC lines derived from individual GSCs. As described herein, GSCs possess fundamental stem cell properties such as clonogenicity, multipotentiality, and self-renewal. GSCs are similar to MSCs isolated from bone marrow based on their morphology, antigen expression pattern, and differentiation potential. GSCs exhibit a substantially expanded life span (>60 population doublings), however, when compared with adult MSCs derived from bone marrow, which normally produce approximately 35 population doublings. GSCs are negative for germ cell specific markers such as Vasa and Dazl, thus representing a new cell population different from germ cells. The cells described herein have the capacity to self renew and differentiate into cells from diverse tissue types, including adipogenic cells, osteogenic cells, chondrogenic, neurogenic cells, and cardiogenic cells. GSCs are easily expandable to therapeutic amounts, making GSCs useful for regenerative medicine. GSCs also can be modified such that the cells can produce one or more polypeptides or other therapeutic compounds of interest.

### Populations and Clonal Lines of GSCs

Purified populations of GSCs can be obtained from an adult human testis sample. As used herein, “purified” means that at least 90% (e.g., 91, 92, 93, 94, 95, 96, 97, 98, or 99%) of the cells within the population are GSCs. As used herein, “GSCs” refers to adult human cells that are positive for CD44, CD105, CD166, CD73, CD90, and STRO-1, negative for CD34, CD45, and HLA-DR, and do not express Vasa, Dazl, and Sox2. “GSC population” refers to the primary culture obtained from the human testis sample and uncloned progeny thereof. “Clonal line” refers to a cell line derived from a single cell. As used herein, a “cell line” is a population of cells able to renew themselves for extended periods of times in vitro under appropriate culture conditions. The term “line,” however, does not indicate that the cells can be propagated indefinitely. Rather, clonal lines described herein typically can undergo 30 to 40 (e.g., 35) doublings before senescing.

Typically, a GSC population is obtained from an adult human testis sample by isolating viable cells from the tissue and then purifying GSCs from the viable cells by adherence to a fibronectin-coated substrate. Preferably, GSCs are purified from an adult human testis sample that is less than 48 hours old (e.g., immediately following biopsy or up to 48 hours after biopsy). Typically, GSCs can be obtained from a small block of testis tissue, e.g., a block of testis tissue that is about 100 to 500 mg or 3-10 mm². The testis tissue can be physically disrupted or subjected to enzymatic digestion to aid in the isolation of viable cells. Any method of physical disruption or enzymatic digestion can be used, provided that the method leaves at least 60%, 70%, 80%, 90%, 95%, 98%, or 99% of the cells in the tissue viable, as determined by trypan blue exclusion. Physical disruption can include crushing, shearing, mincing, dicing, chopping, macerating or the like of the tissue. Enzymatic digestion of the tissue can be performed using one or more tissue-digesting enzymes, including one or more of matrix metalloproteases (e.g., a collagenase such as Type II collagenase), neutral proteases (e.g., dispase), mucolytic enzymes, papain, and serine proteases (e.g., trypsin, chymotrypsin, or elastase). Serine proteases can be inhibited by alpha 2 microglobulin in serum and therefore the medium used for digestion is usually serum-free. EDTA and DNase are commonly used in enzyme digestion procedures to increase the efficiency of cell recovery.

Viable cells can be recovered from the tissue sample by centrifugation then washed (e.g., with a saline solution) and plated on a solid substrate (e.g., a plastic culture device such as a chambered slide or culture flask) coated with fibronectin, using a standard growth medium with 10% serum (e.g., DMEM high glucose with 10% serum). GSCs attach to the surface of the solid substrate while other cells, including spermatogonial cells, do not and can be removed with washing.

Clonal lines of GSCs can be established by plating the cells at a high dilution and using cloning rings (e.g., from Sigma) to isolate single colonies originating from a single cell. Cells are obtained from within the cloning ring using trypsin then re-plated in one well of a multi-well plate (e.g., a 6-well plate). After cells reach >60% confluency (e.g., >70% confluency), the cells can be transferred to a larger culture flask for further expansion.

GSC can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

GSCs can be immunophenotypically characterized using known techniques. For example, the cells can be fixed (e.g., in paraformaldehyde), permeabilized, and reactive sites blocked (e.g., with serum albumin), then incubated with an antibody having binding affinity for a cell surface antigen such as CD34, CD44, CD45, CD73, CD90, CD105, CD166, STRO-1, SSEA-4, or HLA-DR, or any other cell surface antigen. The antibody can be detectably labeled (e.g., fluorescently or enzymatically) or can be detected using a secondary antibody that is detectably labeled. In some embodiments, the cell surface antigens on GSCs can be characterized using flow cytometry and fluorescently labeled antibodies. For example, for flow cytometry, the GSCs can be detached from the tissue culture device and resuspended in a culture medium with a buffer (e.g., MEM plus HEPES) and bovine serum albumin (e.g., 2% BSA), then incubated with a fluorescently labeled antibody having binding affinity for a cell surface antigen.

GSCs also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining).

As described herein, GSCs generally are positive for the cell surface markers CD44, CD105, CD166, CD73, and STRO-1, negative for the cell surface markers CD34, CD45, and HLA-DR, and do not express Vasa, Dazl, and Sox2. As used herein, the phrase “do not express” indicates that mRNA was not detected as compared with suitable positive and negative controls processed and analyzed under similar conditions. GSCs also can be positive for SSEA-4. Clonal lines of GSC (GSC-cs) have a cell surface profile that differs from GSC in that the clones are generally negative for CD90, while GSC are positive for CD90. In addition, a greater percentage of GSC-cs are positive for SSEA-4 and CD34. This suite of cell surface markers, including CD34, CD45, CD73, CD90, CD105, CD166, STRO-1, and HLA-DR, and expression profile for Vasa, Dazl, Oct4, Nanog, and Sox2 can be used to identify GSCs, and to distinguish GSCs from other stem cell types. Because the GSCs express CD73 and CD105, they have MSC-like characteristics. GSCs can be distinguished from MSC, e.g., bone marrow-derived adult MSCs, however, by expression of Oct4 and Nanog. GSCs express Oct4 and Nanog, pluripotent markers typically expressed on embryonic stem cells, while MSCs do not express Oct4 and Nanog. GSCs can be further distinguished from MSC by their substantially expanded life span (>60 population doublings) when compared to bone marrow-derived adult MSCs, which normally produce approximately 35 population doublings. This may be due to the expression of Oct-4 and Nanog in GSCs. Interestingly, both SSEA-4 and CD34 are stem cell markers that are associated with growth; yet GSC-cs underwent replicative arrest much earlier than GSCs. Moreover, it was observed that lack of CD90 expression and increase of SSEA-4 and CD34 expression correlates with enhanced osteogenic differentiation potential for GSC-cs as compared to GSCs. In the same manner, the lack of expression of CD34 and CD45 identifies the GSCs as non-hematopoietic stem cells. The lack of expression of Vasa and Dazl indicates that the GSCs are not of the germ cell lineage.

GSCs can be cryopreserved by suspending the cells (e.g., 2 million cells) in a cryopreservative such as dimethylsulfoxide (DMSO, typically 10%). After adding cryopreservative, the cells can be frozen (e.g., to −90° C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90° C., they can be placed in the liquid phase of the liquid nitrogen storage tank for long term storage. Cryopreservation can allow for long-term storage of these cells for therapeutic use.

### Differentiation of GSC

GSCs are capable of differentiating into a variety of cells of the mesoderm lineage, including adipogenic cells, osteogenic cells, chondrogenic cells, and cardiogenic cells as well as cells of the ectoderm lineage (e.g., neurogenic cells). As used herein, “capable of differentiating” means that a given cell, or its progeny, can proceed to a differentiated phenotype under the appropriate culture conditions. Differentiation can be induced using one or more differentiation agents, including any chemical, cytokine, protein, peptide, or any other substance that is capable of inducing differentiation of a cell. Non-limiting examples of differentiation agents include without limitation, Ca²⁺, an epidermal growth factor (EGF), a platelet derived growth factor (PDGF), a keratinocyte growth factor (KGF), a transforming growth factor (TGF), cytokines such as an interleukin, an interferon, or tumor necrosis factor, retinoic acid, transferrin, hormones (e.g., androgen, estrogen, insulin, prolactin, triiodothyronine, hydrocortisone, or dexamethasone), sodium butyrate, TPA, DMSO, NMF (N-methyl formamide), DMF (dimethylformamide), or matrix elements such as collagen, laminin, or heparan sulfate.

Determination that a GSC has differentiated into a particular cell type can be assessed using known methods, including measuring changes in morphology and cell surface markers (e.g., by flow cytometry or immunohistochemistry), examining morphology by light or confocal microscopy, or by measuring changes in gene expression using techniques such as PCR or gene-expression profiling.

For example, GSCs can be induced to differentiate into osteogenic cells using an induction medium (e.g., AdvanceSTEM™ Osteogenic Differentiation medium, catalog #SH30881.02 from HyClone or Osteogenic Differentiation medium from Lonza, catalog #PT-3002). Typically, osteogenic induction media contain dexamethasone, L-glutamine, ascorbate, and β-glycerophosphate (Jaiswal et al., J. Biol. Chem. 64(2):295-312 (1997)), and in some embodiments, antibiotics such as penicillin and streptomycin. Osteogenic differentiation can be detected by testing for the presence of osteogenic markers, which include, but are not limited to, osteopontin (OP), osteocalcin (OC), osteonectin (ON), bone sialoprotein, and Distal-less homeobox 5 (DLX5). Osteogenesis also can be detected by using von Kossa stain (Jaiswal et al., supra) and/or alizarin red stain (Wan et al., Chin. J. Traumatatol. 5:374-379 (2002)), which detect the presence of calcium deposits.

GSCs can be induced to differentiate into adipogenic cells using an induction medium (e.g., AdvanceSTEM™ Adipogenic Differentiation Medium from HyClone, catalog #SH30886.02; or Adipogenic Differentiation Medium, catalog #PT-3004, from Lonza). Typically, adipogenic differentiation media contain human insulin, L-glutamine, dexamethasone, indomethacin, and 3-isobutyl-1-methyl-xanthine. For example, GSCs can be cultured in Adipogenesis Differentiation Medium for 3 days (at 37° C., 5% CO₂), followed by 1 day of culture in Adipogenesis Maintenance Medium (catalog #PT-3102A, from Lonza) containing human insulin and L-glutamine. After 3 complete cycles of induction/maintenance, the cells can be cultured for an additional 7 days in Adipogenesis Maintenance Medium, replacing the medium every 2-3 days.

Adipogenic cells contain lipid filled liposomes that can be visualized with Oil Red stain (Conget and Minguell, J. Cellular Physiology 181:67-73, (1999)). Such cells also contain trigycerides, which fluoresce green with Nile Red stain (Fowler and Greenspan, Histochem. Cytochem. 33:833-836 (1985)). Adipogenic differentiation also can be assessed by testing for the presence of adipogenic transcription factors PPARy2 (peroxisome proliferator activated receptor gamma) and/or CEBPα (CCAAT/enhancer binding protein alpha), or for lipoprotein lipase by methods such as immunohistochemistry and RT-PCR.

GSC can be induced to differentiate into chondrogenic cells using an induction medium (e.g., AdvanceSTEM™ Chondrogenic Differentiation Medium from HyClone, catalog #SH30889.02, or Chondrogenic Differentiation Medium from Lonza, catalog #PT-3003). Typically, chondrogenic differentiation media contain dexamethasone, ascorbate, sodium pyruvate, proline, L-glutamine, and TGF-(33. Chondrogenic cells contain sulfate proteoglycans that can be visualized with Alcian Blue stain. Such cells also contain Type II collagen. Chondrogenic differentiation also can be assessed by testing for the presence of aggrecan and/or link protein.

GSC can be induced to differentiate into neurogenic cells using an induction medium. Typically, neurogenic differentiation media contain growth factors such as basic fibroblast growth factor (bFGF) and EGF; or sonic hedgehog (SHH), FGF, and bFGF; EGF or brain derived neurotrophic factor (BDNF), and glial derived neurotrophic factor (GDNF)). Retinoic acid (RA) and ascorbic acid also can be included in a neurogenic differentiation medium. For example, GSCs can be cultured on fibronectin or Matrigel™ coated plates in the presence of media containing putrescine and growth factors (bFGF and EGF, or SHH, FGF8, and bFGF) for 12 days, wherein RA is added to the cultures from days 10-12. After incubating in such media for 12 days, the media can be replaced with media containing EGF or BDNF, GDNF, and ascorbic acid, and the cells incubated for an additional 14 days. Neurogenic differentiation can be assessed by testing for the presence of nestin, class III beta-tubulin (tubulin β-4), glial fibrillary acidic protein (GFAP), neuro-specific enolase (NSE), microtubule-sasociated protein 2 (MAP2), or galactocerebroside (GalC).

GSC can be induced to differentiate into cardiogenic cells using an induction medium. Typically, cardiogenic differentiation media contain 5-AZA-2′-deoxycytidine (Aza). Cardiogenic differentiation can be assessed by testing for the presence of cardiac markers such as demin, troponin I, troponin T, or atrial natriuretic factor (ANF).

In some embodiments, the GSCs can be cultured or seeded onto bio-compatible scaffolds. Such scaffolds can act as a framework that supports the growth of the cells in multiple layers. Scaffolds can be molded into the desired shape for facilitating the development of tissue types. For example, the cells can be seeded on a scaffold and induced to differentiate into osteogenic cells or chondrogenic cells as discussed above.

Typically, the scaffold is formed from collagen or a polymeric material. Biodegradable scaffolds are particularly useful such that after implantation into an animal, the scaffold can be absorbed into the animal matter over time. Suitable polymeric scaffolds can be formed from monomers such as glycolic acid, lactic acid, propyl fumarate, caprolactone, hyaluronan, hyaluronic acid, and combinations thereof. Other scaffolds can include proteins, polysaccharides, polyhydroxy acids, polyorthoesters, polyanhydrides, polyphosphazenes, synthetic polymers (particularly biodegradable polymers), and combinations thereof. The scaffold also can include hormones, growth factors, cytokines, and morphogens (e.g., retinoic acid), desired extracellular matrix molecules (e.g., fibronectin), or other materials (e.g., DNA, viruses, other cell types, etc.). See, e.g., U.S. Pat. No. 7,470,537.

The GSCs can be loaded into the scaffold by soaking the scaffold in a solution or suspension containing the GSCs, or the GSCs can be infused or injected into the scaffold. In other embodiments, a hydrogel can be formed by crosslinking a suspension including the desired polymer and the GSCs, allowing the GSCs to be dispersed throughout the scaffold. To direct the growth and differentiation of the desired structure, the scaffold containing the GSCs can be cultured ex vivo in a bioreactor or incubator, as appropriate. In other embodiments, the scaffold containing the GSCs can be implanted within a host animal directly at the site in which it is desired to grow the tissue or structure. In still another embodiment, the scaffold containing the GSCs can be engrafted on a host (typically an animal such as a pig), where it can grow and mature until ready for use.

### Modified Populations of GSCs

GSCs can be modified such that the cells can produce one or more polypeptides or other therapeutic compounds of interest. To modify the isolated cells such that a polypeptide or other therapeutic compound of interest is produced, the appropriate exogenous nucleic acid must be delivered to the cells. In some embodiments, the cells are transiently transfected, which indicates that the exogenous nucleic acid is episomal (i.e., not integrated into the chromosomal DNA). In other embodiments, the cells are stably transfected, i.e., the exogenous nucleic acid is integrated into the host cell's chromosomal DNA. The term “exogenous” as used herein with reference to a nucleic acid and a particular cell refers to any nucleic acid that does not originate from that particular cell as found in nature. In addition, the term “exogenous” includes a naturally occurring nucleic acid. For example, a nucleic acid encoding a polypeptide that is isolated from a human cell is an exogenous nucleic acid with respect to a second human cell once that nucleic acid is introduced into the second human cell. The exogenous nucleic acid that is delivered typically is part of a vector in which a regulatory element such as a promoter is operably linked to the nucleic acid of interest.

Cells can be engineered using a viral vector such as an adenovirus, adeno-associated virus (AAV), retrovirus, lentivirus, vaccinia virus, measles viruses, herpes viruses, or bovine papilloma virus vector. See, Kay et al. Proc. Natl. Acad. Sci. USA 94:12744-12746 (1997) for a review of viral and non-viral vectors. A vector also can be introduced using mechanical means such as liposomal or chemical mediated uptake of the DNA. For example, a vector can be introduced into GSCs by methods known in the art, including, for example, transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter.

A vector can include a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

GSCs also can have a targeted gene modification. Homologous recombination methods for introducing targeted gene modifications are known in the art. To create a homologous recombinant GSC, a homologous recombination vector can be prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, Cell 51:503 (1987); Bradley, Curr. Opin. Bio/Technol. 2:823-29 (1991); and PCT Publication Nos. WO 90/11354, WO 91/01140, and WO 93/04169.

### Compositions and Articles of Manufacture

This document also features compositions and articles of manufacture containing purified populations of GSC or clonal lines of GSC. In some embodiments, the purified population of GSC or clonal line is housed within a container (e.g., a vial or bag). In some embodiments, the clonal lines have undergone at least 3 doublings in culture (e.g., at least 4, 5, 6, 7, 8, 9, 10, 15, 20, 25, 30, 35, or 40 doublings). In other embodiments, a culture medium (e.g., DMEM with high glucose) is included in the composition or article of manufacture. In still other embodiments, the composition or article of manufacture can include one or more cryopreservatives. In some embodiments, GSCs or clonal lines can be formulated as pharmaceutical compositions.

Generally, a pharmaceutical composition includes a pharmaceutically acceptable carrier, additive, or excipient and is formulated for an intended mode of delivery, e.g., intravenous, subcutaneous, or intramuscular administration, or any other route of administration described herein. For example, a pharmaceutical composition for intravenous administration can include a physiological solution, such as physiological saline and water, Ringers Lactate, dextrose in water, Hanks Balanced Salt Solution (HBSS), Isolyte S, phosphate buffered saline (PBS), or serum free cell media (e.g., RPMI). Pharmaceutical compositions also can include, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of a composition can be adjusted with acids or bases, such as hydrochloric acid or sodium hydroxide.

Pharmaceutical compositions should be stable under the conditions of processing and storage and must be preserved against potential contamination by microorganisms such as bacteria and fungi. Prevention of contamination by microorganisms can be achieved by various antibacterial and antifungal agents, e.g., antibiotics such as aminoglycosides (e.g., kanamycin, neomycin, streptomycin, and gentamicin), ansaycins, and quinalones.

The pharmaceutical composition can be formulated to include one or more additional therapeutic agents. For example, a composition can be formulated to include one or more growth factors and/or one or more anti-inflammatory agents, including non-steroidal anti-inflammatory drugs, dexamethasone or other types glucocorticoid steroids, PDGF, EGF, fibroblast growth factor-2, stem cell factor, a bone morphogenic protein (BMP) such as BMP-2 or BMP-7, methylsulfonylmethane (MSM), glucosamine, or chondroitin sulfate.

Purified populations of GSC or clonal GSC lines can be combined with packaging material and sold as a kit. The packaging material included in a kit typically contains instructions or a label describing how the purified populations of GSC or clonal lines can be grown, differentiated, or used. Components and methods for producing such kits are well known.

An article of manufacture or kit also can include one or more reagents for characterizing a population of GSCs or a clonal GSC line. For example, a reagent can be a nucleic acid probe or primer for detecting expression of a gene such as Oct4, Nanog, Sox2, vimentin, Vasa, or Dazl. Such a nucleic acid probe or primer can be labeled, (e.g., fluorescently or with a radioisotope) to facilitate detection. A reagent also can be an antibody having specific binding affinity for a cell surface marker such as CD44, CD45, SSEA-4, CD34, CD73, CD90, CD105, CD166, STRO-1, or HLA-DR. An antibody can be detectably labeled (e.g., fluorescently or enzymatically). Other components, such as a scaffold (e.g., a scaffold composed of collagen), also can be included in a composition or article of manufacture. The scaffold can be seeded with GSCs as described above.

### Methods of Using GSCs

Populations of GSCs or clonal lines of GSC can be used to treat subjects having a variety of disorders or injuries, including atrophic nonunion, bone fractures, autoimmune diseases, spinal cord injuries, stroke, and diabetes, as well as to repair cartilage and spinal discs (e.g., degenerative discs and meniscus). The GSCs or clonal lines can be delivered to a subject in various ways as appropriate to deliver stem cells, including, but not limited to oral or parenteral routes of administration such as intravenous, intramuscular, intraperitoneal, subcutaneous, intrathecal, intraarterial, or nasal. In some embodiments, two or more routes of administration can be used to deliver the stem cells. In other embodiments, the cells are delivered to a site of the injury. For example, a scaffold containing the GSCs can be delivered to the site of a cartilage, bone, or disc injury.

Effective amounts of GSCs or clonal lines can be determined by a physician, taking into account various factors such as overall health status, body weight, sex, diet, time and route of administration, other medications, and any other relevant clinical factors. In some embodiments, between 500,000 and 2,000,000 (e.g., 500,000 to 1,000,000; 500,000 to 750,000; 750,000 to 1,000,000; 750,000 to 2,000,000; 750,000 to 1,500,000; 1,000,000 to 2,000,000; 1,000,000 to 1,500,000; or 1,500,000 to 2,000,000) stem cells/kg weight of the subject can be delivered to the subject in total. In some embodiments, about 1.2×10⁶ GSCs/kg weight of the subject are delivered to the subject.

In some embodiments, between 500,000 and 500,000,000 (e.g., 5×10⁵, 6×10⁵, 7×10⁵ 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, or 5×10⁸) GSCs/kg weight of the subject can be delivered to the subject in total.

In some embodiments, GSCs are delivered to the subject only once. In some embodiments, multiple (e.g., two, three, four, five, six, seven, eight, nine, 10, 11, 12, 13, 14, 15, or 20 or more) deliveries are made. For example, multiple deliveries of GSCs can be made over the course of several (e.g., two, three, four, five, six, seven, eight, nine, 10, 14, 21, 28, or 31 or more) consecutive days (e.g., one delivery each day for seven days or one delivery every other day for seven days). GSCs can be delivered to a subject for several months (e.g., one delivery per month for six months, or one delivery per week for two months).

GSCs can be delivered to a subject at various time points after injury (e.g., a cartilage injury). For example, the cells can be delivered immediately following an injury (e.g., from 1 to 8 such as 1.5, 2, 2.5, 3, 3.5, 4, 4.5, 5, 5.5, 6, 6.5, 7, 7.5, or 8 hours after the injury occurs). The cells can be delivered to a subject less than 10 (e.g., 9, 8, 7, 6, 5, 4, 3, 2, or 1) days after an injury occurs. The cells can be delivered to a subject less than 6 (e.g., 5, 4, 3, 2, or 1) weeks after an injury occurs. In some embodiments, GSCs can be delivered to a subject up to 10 years (e.g., 9, 8, 7, 6, 5, 4, 3, 2, or 1) years after an injury occurs. The compositions and methods described herein can be used at any time following an injury or during the course of a chronic injury.

It is understood that regardless of the site, combination of sites, route of administration, combination of routes, a therapeutically effective amount of GSCs (or a composition that includes the GSCs) is delivered to the subject. As used herein, an “effective amount” or “therapeutically effective amount” of a composition or GSCs is the amount that is sufficient to provide a beneficial effect to the subject to which the composition or cells are delivered. The effective amount can be the amount effective to achieve an improved survival rate, a more rapid recovery, an improvement in the quality of life, or an improvement or elimination of one or more symptoms associated with a subject's condition.

The efficacy of a given treatment in treating a particular disorder or an injury can be defined as an improvement of one or more symptoms of the disorder or injury by at least 5% (e.g., at least 10%, at least 15%, at least 20%, at least 25%, at least 30%, at least 40%, at least 50%, at least 55%, at least 60%, at least 65% or more). In some cases, efficacy of a treatment with GSCs can be determined from the stabilization of one or more worsening symptoms associated with the injury (i.e., the treatments curtail the worsening of one or more symptoms of the injury).

GSCs or pharmaceutical compositions containing GSCs can be administered to a subject in combination with another treatment, e.g., a treatment for a bone injury. For example, the subject can be administered one or more additional agents that provide a therapeutic benefit to the subject who has a bone injury. Additional therapeutic agents include, e.g., growth factors and/or anti-inflammatory agents (e.g., non-steroidal anti-inflammatory drugs, dexamethasone or other types glucocorticoid steroids, PDGF, EGF, fibroblast growth factor-2, stem cell factor, a bone morphogenic protein (BMP) such as BMP-2 or BMP-7, methylsulfonylmethane (MSM), glucosamine, or chondroitin sulfate. The GSCs or pharmaceutical compositions and the one or more additional agents can be administered at the same time or sequentially.

The invention is further described in the following examples, which do not limit the scope of the invention described in the claims.

### EXAMPLES

### Example 1

### Expansion and Growth Kinetics of GSCs

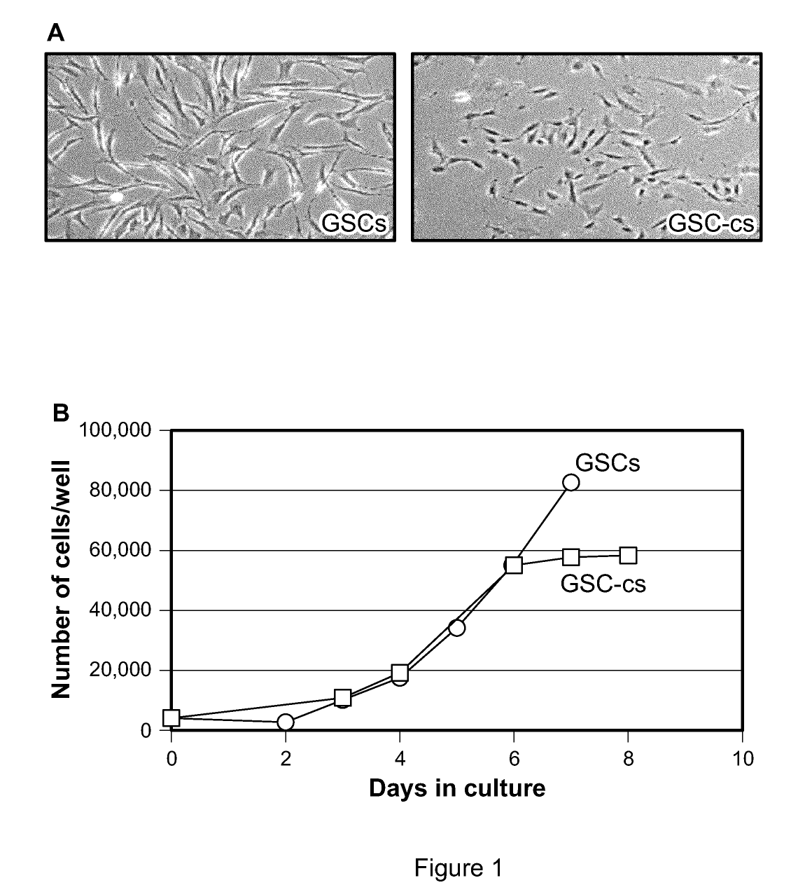

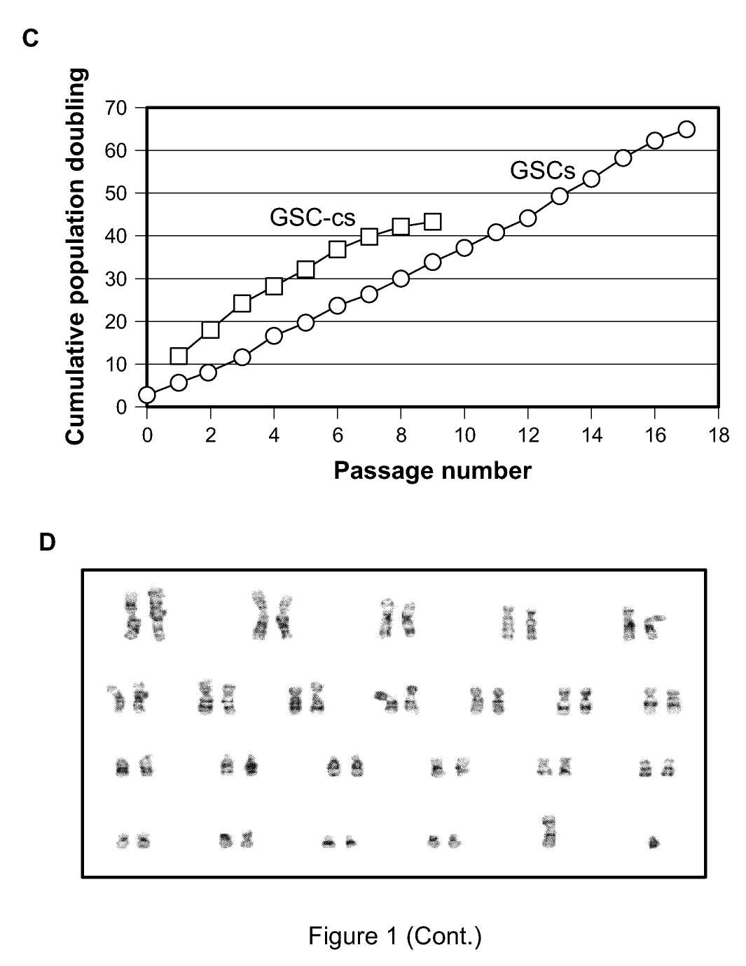

In order to isolate a novel stem cell population, cells were isolated from tissue obtained from testicular biopsies (DV Biologics LLC). Biopsies were obtained from six 22-47 years old donors after informed consent as approved by an institutional regulatory board (IRB) (DV Biologics LLC). Tissue was digested with 0.1% Collagenase type II (Sigma Aldrich) solution for 10-20 min at 37° C. After filtration through a 40 μm strainer and centrifugation at 1,500×g for 10 min at 4° C., cells were counted and plated on 10 cm dishes coated with Fibronectin 10 μg/mL (Sigma Aldrich). Cells were cultured in DMEM high glucose with GlutaMax (Gibco) supplemented with 10% FBS (Hyclone), fibroblast growth factor 2 (FGF2) (10 ng/ml), glial cell derived neurotrophic factor (GDNF) (10 ng/ml) (Invitrogen) and penicillin/streptomycin (Gibco). After 3 days, non adhering cells (e.g., spermatogonial cells and dying cells) were discarded and attached cells were cultured and expanded. Attached cells were fed every 3-4 days. Under these conditions, cells reached 70% confluency after 7-10 days and exhibited MSC-like morphology (FIG. 1A). At 70-80% confluency, cells were detached using TrypZean solution (Sigma) and re-plated on flasks without coating at a density of 1,000 cells/cm2.

Cloning efficiency of the cells was assessed as follows. For colonies, cells were plated at a density of 150 cells/10 cm dish in DMEM high glucose with GlutaMax supplemented as described above. After 17 days, cells were fixed and stained in a 9% Crystal Violet Methanol solution for 1 min. Cloning efficiency was estimated as the percentage of cells which generated clones from the total cell number/dish. For cell cloning, 100 cells/10 cm dish were seeded. Selected clones were isolated using cloning rings (Sigma) and detached with Trypsin solution. Each clone was re-plated in one well of a 6-well plate. After reaching 70% confluency, cells were seeded in 75 cm2 flasks for further expansion. Clonogenic efficiency of the whole cell population was 35+1.8% (n=5). In total, 7 clones (GSC-cs) were collected and every clone was successfully expanded. GSC-sc exhibited a statistically significant (p<0.001) decrease in clonogenic efficiency (7+0.6%, n=5) in comparison with the starting population.

To generate growth curves, cells (GSC or GSC-cs) were plated onto 24-well plates at a density of 4,000 cells/well and counted in triplicates from days 3 to 8. Exponential interval of the growth curve was used to calculate doubling time as described by Berthon et al., In Vitro Cell Dev Biol. 28A (11-12):716-724 (1992). For population doublings (PD), cells were cultured on 25 cm2 flasks, harvested, counted and re-plated when they reached 70-80% confluency. Cell culture was terminated when the cell population failed to double after 2 weeks of culture. Population doubling was calculated using the formula PD=[log 10(N1)−log 10(N0)/log 10(2) where N1 is cell number at harvesting and NO is cell number at plating as described Cristofalo et al., Proc. Natl. Acad. Sci. USA, 95:10614-10619 (1998). Doubling times were similar for both populations (33.8±6.5 h GSC and 32.4±4.4 h GSC-cs) (FIG. 1B). However, the proliferative capacity of GSC-cs was markedly reduced in comparison to that of GSCs (FIG. 1C). GSCs propagated for 17 passages with at least 64 population doublings (FIG. 1C) and were easily expanded to therapeutically necessary amounts by passage 3 (>2.0×10⁸). Karyotype analysis of GSC-cs was performed by Cell Line Genetics using standard cytogenetic protocols and G-banding of karyograms produced from at least 20 metaphases. GSC-cs are diploid cells without chromosomal aberrations as determined by karyotype analysis (FIG. 1D).

### Example 2

### Characterization of GSCs

In order to characterize GSCs, flow cytometry, immunocytochemistry, and RT-PCR was performed. For immunocytochemistry, cells were fixed in 4% paraformaldehyde (PFA) and stored at 4° C. After permeabilization in 0.1% of Triton X-100 (Promega) and blocking in 2% BSA (Sigma), primary antibody diluted in blocking buffer was applied overnight at 4° C. Staining for SSEA-4 was performed without permeabilization in 0.1% Triton X-100 solution. Cells were incubated with secondary antibody in blocking buffer for 1 hour at room temperature. Cells were counterstained with DAPI (Molecular probes) and mounted in Fluoromount-G (Southern Biotech). Primary antibodies used were: oct3/4 clone H-134 (Santa Cruz Biotech), nanog (ReproCell), SSEA-4 (Millipore), vimentin (Dako), LHR (Millipore), and 3β HSD (Santa Cruz Biotech). Secondary antibodies Alexa 488 and Alexa 594 (Molecular probes) were used. For negative controls, incubation without primary antibody and with corresponding specific non-immune immunoglobulins (Santa Cruz Biotech) were used. Staining was analyzed using an Olympus IX81 inverted microscope and SlideBook software.

For flow cytometry, cells were detached, filtered through a 40m strainer, pelleted, resuspended in MEM+HEPES (Gibco) with 2% BSA and counted. Directly conjugated antibodies were: CD105, CD166, CD90, CD44, CD45, CD34, CD11b, CD19, HLA-ABC, HLA-DP DQ DR (Serotec), CD133 (Miltenyi Biotech) LIN, CD73 (BD Pharmingen). For anti-SSEA-4 and anti-STRO-1 staining (Millipore), secondary antibody goat anti-mouse IgG+IgM-APC (Jackson Immunoresearch) was used. After staining, cells were fixed with 4% paraformaldehyde and analyzed using CyAn ADP Analyzer 9 color (Beckman Coulter). Histograms were generated by using Flowjo software (Treestar Inc.).

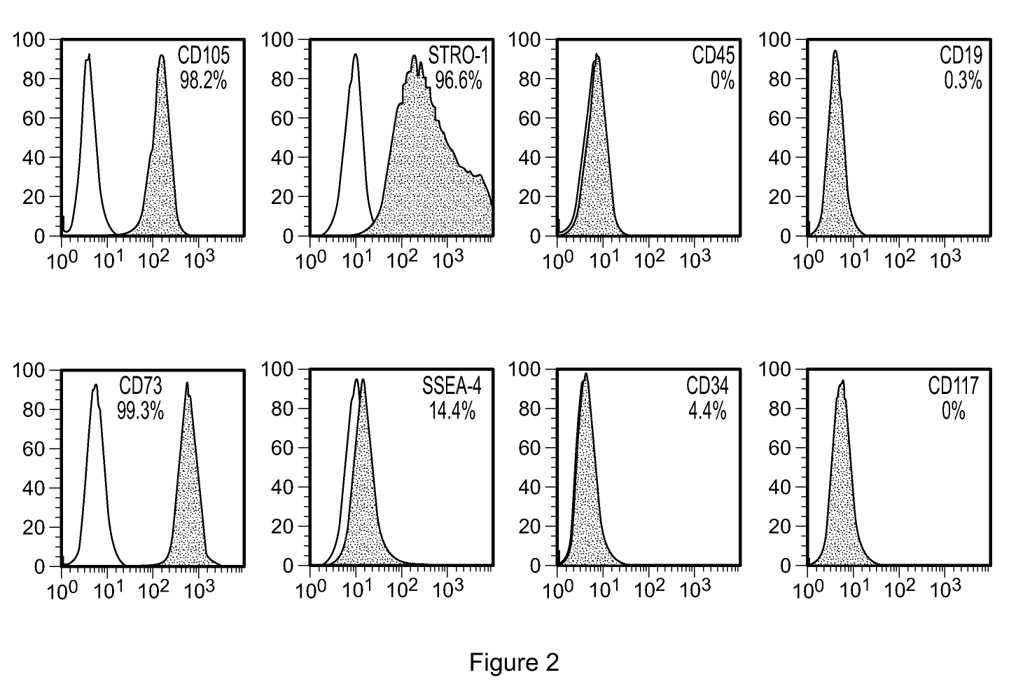

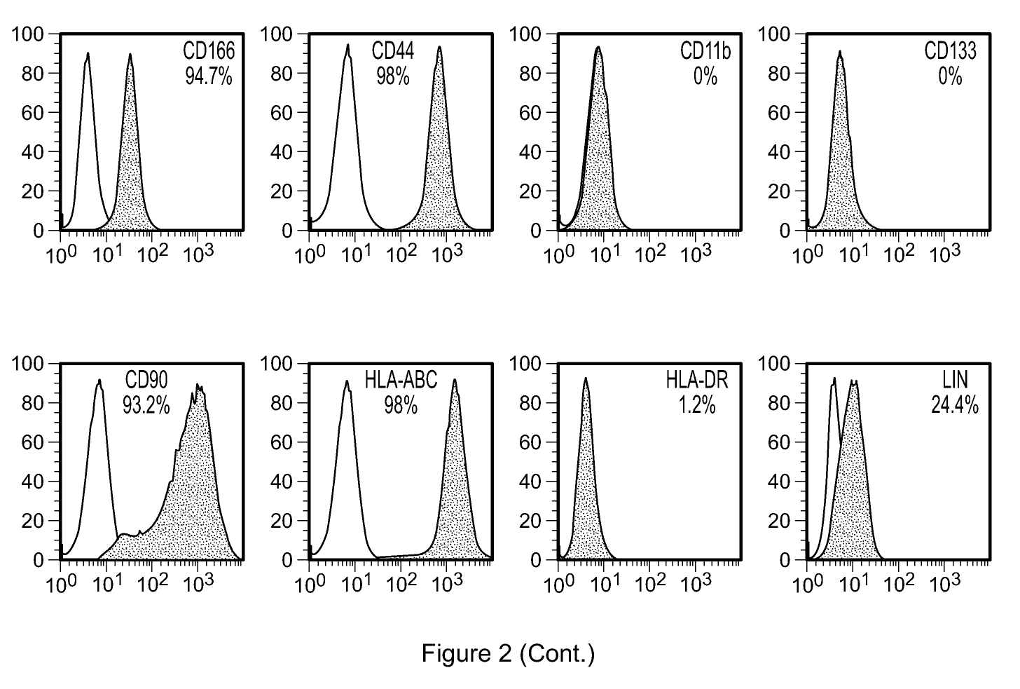

Flow cytometry analysis revealed that cells expanded in culture show characteristics typical of MSCs isolated from bone marrow in accordance with the International Society for Cellular Therapy minimum criteria for defining MSCs [see Dominici et al. Cytotherapy 8(4):315-317 (2006)]. GSCs were positive for CD105, CD73, and CD166 and negative for CD34, CD45, HLA-DR, CD11b and CD19 (FIG. 2). In addition, GSCs expressed high levels of CD44, CD90 and STRO-1 which are expressed on MSCs [see Gonzalez et al. Biochem Biophys Res Commun 362(2) 491-497 (2007); Ho et al. Cytotherapy 10(4):320-330 (2008)].

Interestingly, a small percentage of GSCs express the pluripotent stem cell marker stage-specific embryonic antigen 4 (SSEA-4) (FIG. 2). Based on morphology similar to the several distinct cell types described in MSCs, GSCs are also a heterogeneous population. When comparing GSCs with GSC-cs, their morphology (FIG. 1A) and antigen expression (Table 1) were different. Results in Table 1 are based on the characterization of surface antigen expression of the whole population of GSC and GSC-clone 9, which were stained simultaneously and subjected to flow-cytometric analysis. Both populations expressed antigens found on MSCs, including CD105, CD73, CD166, CD44, STRO-1, SSEA-4, and were largely negative for hematopoietic markers CD45, CD34, and HLA-DR with few exceptions for GSC-cs. GSC-cs were mostly negative for CD90 (Thy-1), and had a higher expression of SSEA-4 and CD34 as compared to GSCs. Percent of positive cells were calculated as percent of stained cells minus percent of positive cells in the corresponding isotype control. GSCs had a morphology similar to MSCs while GSC-cs were much smaller and had less processes.

TABLE 1

| | GSC | | GSC | |
|---|---|---|---|---|
| | whole population | | Clone #9 | |
| | % positive | Mean | % positive | Mean |
| Antigen | cells | fluorescence | cells | fluorescence |
| CD105 | 98.4 | 136 | 98.1 | 95 |
| CD73 | 99.3 | 448 | 97.9 | 323 |
| CD166 | 94.9 | 32 | 85 | 27 |
| CD90 | 95 | 691 | 3.3 | 64 |
| CD44 | 99.8 | 642 | 98.7 | 464 |
| STRO-1 | 98.6 | 323 | 98.9 | 642 |
| SSEA-4 | 16.1 | 33 | 46.8 | 34 |
| CD133 | 0.1 | 55 | 0.8 | 22 |
| CD117 | 0.1 | 24 | 1 | 22 |
| CD45 | 1.1 | 46 | 2.2 | 29 |
| CD34 | 4.6 | 27 | 14.4 | 25 |
| CD11b | 1.4 | 48 | 2.3 | 18 |
| CD19 | 0.5 | 14 | 2.3 | 18 |
| HLA-ABC | 99.8 | 1372 | 98.8 | 2041 |
| HLA-DR | 1.4 | 22 | 3.3 | 18 |

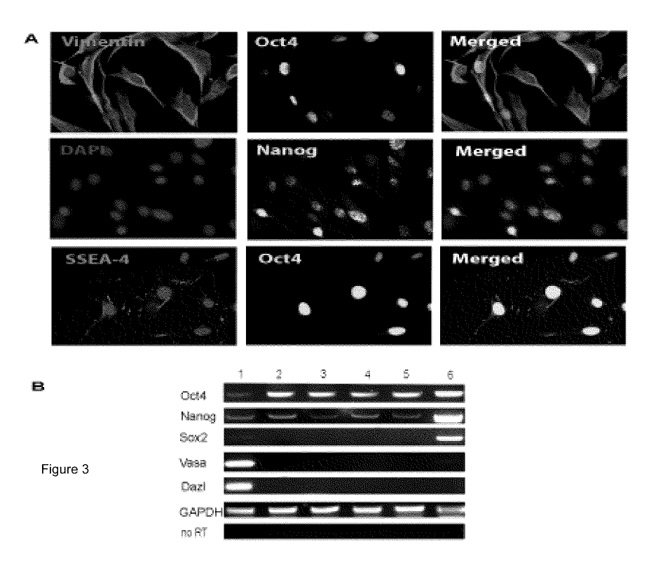

Immunocytochemistry analysis demonstrated that GSCs express the pluripotent markers Oct 4, Nanog, and SSEA-4 (FIG. 3A). To analyze gene expression profile, cells were collected in RLT buffer (Qiagen) and stored at −80° C. until RNA extraction. NT2 cells used as a control for pluripotency genes and were purchased from the ATCC (Manassas, Va., Catalog No. CRL-1973). Total RNA was isolated with the RNeasy Plus kit (Qiagen). 200-300 ng RNA was reverse transcribed using ThermoScript (Invitrogen). Table 2 shows the gene specific primers that were used in both end point and real time PCR reactions.

TABLE 2
RT-PCR PRIMERS

| | | SEQ | | SEQ |
|---|---|---|---|---|
| | Forward | ID | Reverse | ID |
| Gene | (sequence 5′ to 3′) | NO: | (sequence 5′ to 3′) | NO: |
| Oct4 | CGACCATCTGCCGCTTTGAG | 1 | CCCCCTGTCCCCCATTCCTA | 2 |
| Nanog | AGCATCCGACTGTAAAGAATCT | 3 | CGGCCAGTTGTTTTTCTGCCACCT | 4 |
| | TCAC | | | |
| Sox2 | CCCCCGGCGGCAATAGCA | 5 | TCGGCGCCGGGGAGATACAT | 6 |
| Daz1 | GGAGCTATGTTGTACCTCC | 7 | GTGGGCCATTTCCAGAGGG | 8 |
| Vasa | AGAAAGTAGTGATACTCAAGG | 9 | TGACAGAGATTAGCTTCTTCAAAA | 10 |
| | ACCAA | | GT | |
| Lipoprotein lipase | GTCCGTGGCTACCTGTCATT | 11 | TGGCACCCAACTCTCATACA | 12 |
| PPAR-y isoform 2 | GTGAAACTCTGGGAGATTCTCC | 13 | CGACATTCAATTGCCATGAG | 14 |
| Osteocalcin | ATGAGAGCCCTCACACTCCTC | 15 | GCCGTAGAAGCGCCGATAGGC | 16 |
| DLX5 | GAGAAGGTTTCAGAAGACTCA | 17 | CTAGAACAGCAAAACACAGTAGT | 18 |
| | GTGA | | C | |
| Aggrecan | AGCCTGCGCTCCAATGACT | 19 | TGGAACACGATGCCTTTCAC | 20 |
| Proteoglycan link | CCTATGATGAAGCGGTGC | 21 | TATCTGGGAAACCCACGAAG | 22 |
| protein | | | | |
| GAPDH | TGAAGGTCGGAGTCAACGGAT | 23 | CATGTGGGCCATGAGGTCCACCAC | 24 |
| | TTGG | | | |

Real-time PCR was performed with a CFX96™ Real Time System and iQ™ SybrGreen Supermix (Bio-Rad Laboratories) to assess the expression of osteocalcin. GAPDH mRNA was used as a control. Each sample was measured in triplicate. End point PCR was conducted in a C1000™ Thermal Cycler (Biorad) using GoTaq^{R} Hot Start Polymerase (Promega) and 1 μl of cDNA product for the analyses of all other genes. “No RT” and “no template” controls were included in each experiment. Student T-Test was done in order to establish statistical differences in induced samples as compared to controls.

RT-PCR experiments confirmed that GSCs express Oct 4 and Nanog but are negative for Sox 2 (FIG. 3B). GSCs also express vimentin, which is a major subunit protein of the intermediate filaments of mesenchymal cells (FIG. 3A). There are possibilities that GSCs are derived from other cell lineages present in testes, namely germ or Leydig. RT-PCR for Vasa and Dazl confirms that GSCs are not of the germ cell lineage (FIG. 3B). Additionally, the GCS are not precursors or adult Leydig cells, based on the negative immunocytochemistry staining for luteinizing hormone (LH) receptor and 3β-hydroxysteroid dehydrogenase [Teerds et al. Biol Reprod. 60:1437-1445 (1999)] (data not shown).

### Example 3

### GSCs Differentiate into Cells of Mesodermal Lineage

The hallmark of MSCs is the ability to differentiate into mesodermal lineage, including adipogenic, osteogenic and chondrogenic lineages. Both GSCs and GSC-cs were induced to the adipogenic, osteogenic, and chondrogenic lineages using standard MSC differentiation protocols as described below. In addition, GSCs were induced to differentiate into cardiogenic cells as described below.

For adipogenic differentiation, cells at passages 1-4 were plated at a density of 4,000 cells/well in 12-well plates in DMEM high glucose with GlutaMax, supplemented as described in Example 1. At 90-100% confluency, cells were switched to adipogenic induction medium according to manufacturer's protocol (Lonza, PT-3004). After 3 days, medium was changed to adipogenic maintenance medium (Lonza) and kept for 1 day. Cycles of 3 days induction+1 day maintenance medium were repeated for 12-19 days. Control cells were kept in DMEM high glucose with GlutaMax, supplemented as described in Example 1. At 12 and 19 days, cells were fixed with 4% PFA and stored at 4° C. until staining Staining was performed using 0.3% Oil Red 0 solution (Sigma). Nuclei were counterstained for 5 min with Gill #2 Hematoxylin (Sigma). For quantitative assay, Oil Red 0 bound to lipid droplets was extracted with 100% Ethanol solution and absorbance was measured at 550 nm with reference wavelength 650 nm. Absorbance measurements of Oil Red 0 release were compared to standard titration curve of corresponding dye. Obtained quantity of dye accumulation/well was normalized to cell number determined by Hoechst 33342 (Molecular probes) staining of nuclei.

For osteogenic differentiation, cells plated on 12-well dishes were switched to osteogenic differentiation medium (HyClone, catalog #SH30877.KT) according to manufacturer's protocol when 90-100% confluent. After 12 and 19 days of induction, cells were fixed in 4% PFA and stained with 2% Alizarin Red S (Sigma). To detect calcium deposit accumulation, Ca-bound Alizarin Red S was extracted in 10% of cetylpyridinium (Sigma) in phosphate buffer (8 mM Na₂HPO₄+1.5 mM KH₂PO₄, Sigma). Alizarin Red S release was measured at 550 nm with reference wavelength 650 nm. Absorbance measurements of Alizarin Red S release were compared to standard titration curve of corresponding dye. Obtained quantity of dye accumulation/well was normalized to cell number determined by Hoechst 33342 (Molecular probes) staining of nuclei.

For chondrogenic differentiation, cells were placed in either control media (i.e., DMEM high glucose) or chondrogenic differentiation medium according to manufactures' protocol (Lonza, catalog #PT-3003). Briefly, 300,000 cells/15 ml tube were pelleted, and control or chondrogenic differentiation medium was added. After 28 days, pellets were fixed with 4% PFA. Pellets were sunk in 25% sucrose solution and frozen embedded 48 hours after in OCT compound (Sakura Finetek). Pellets were sectioned at 10 μm and stained with 1% Alcian Blue(Sigma) and counter stained with nuclear fast red (Sigma) using standard protocols.

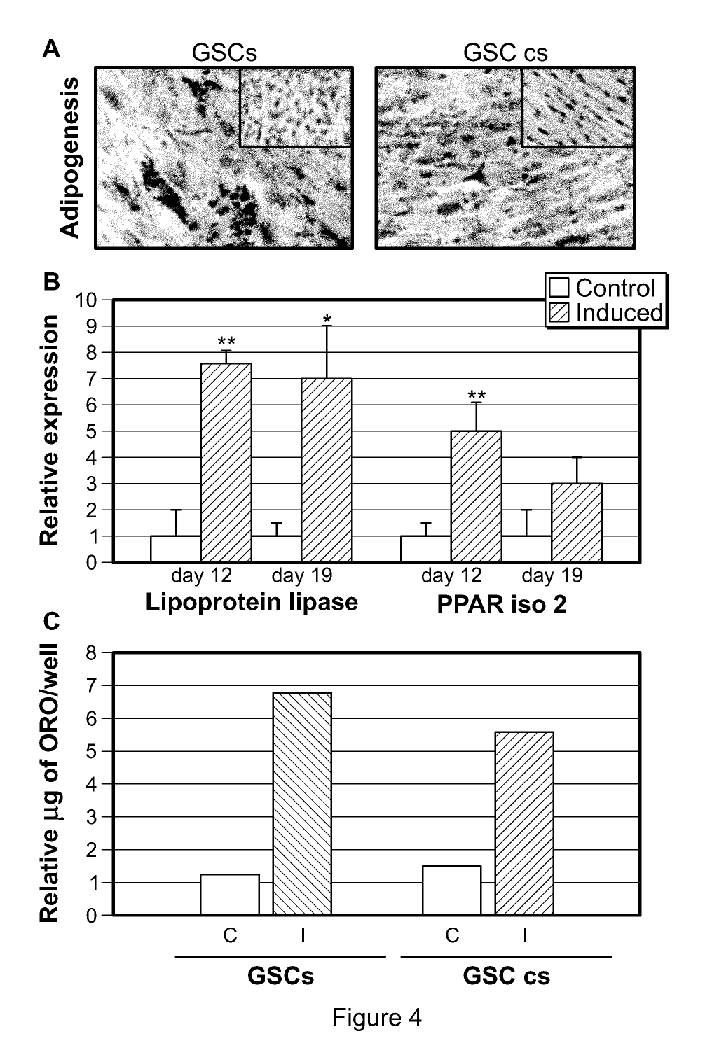

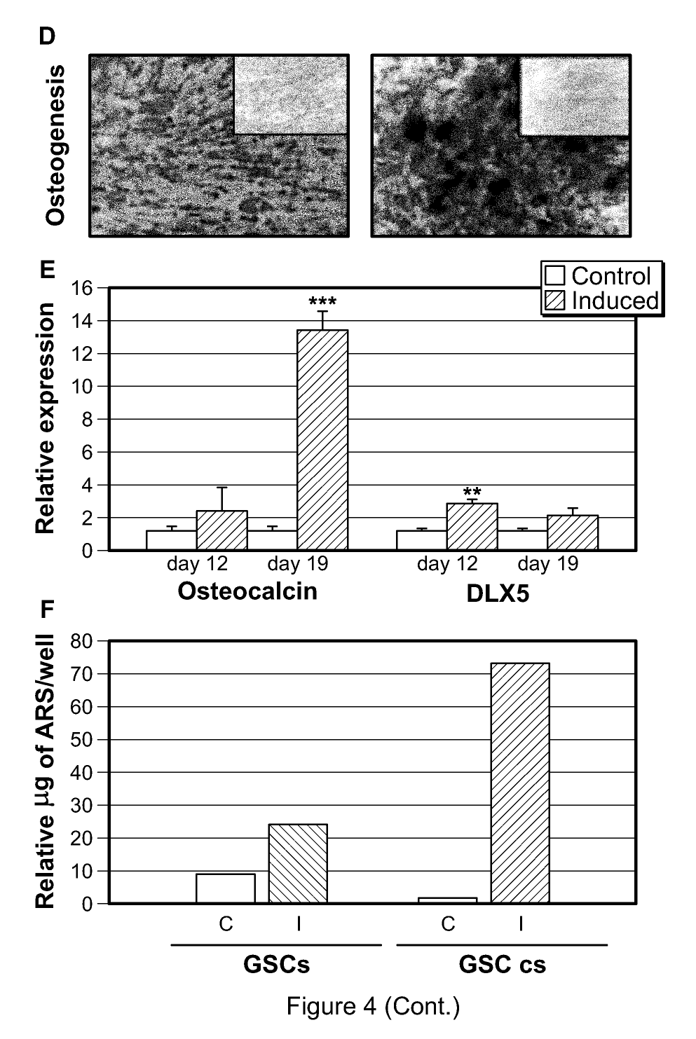

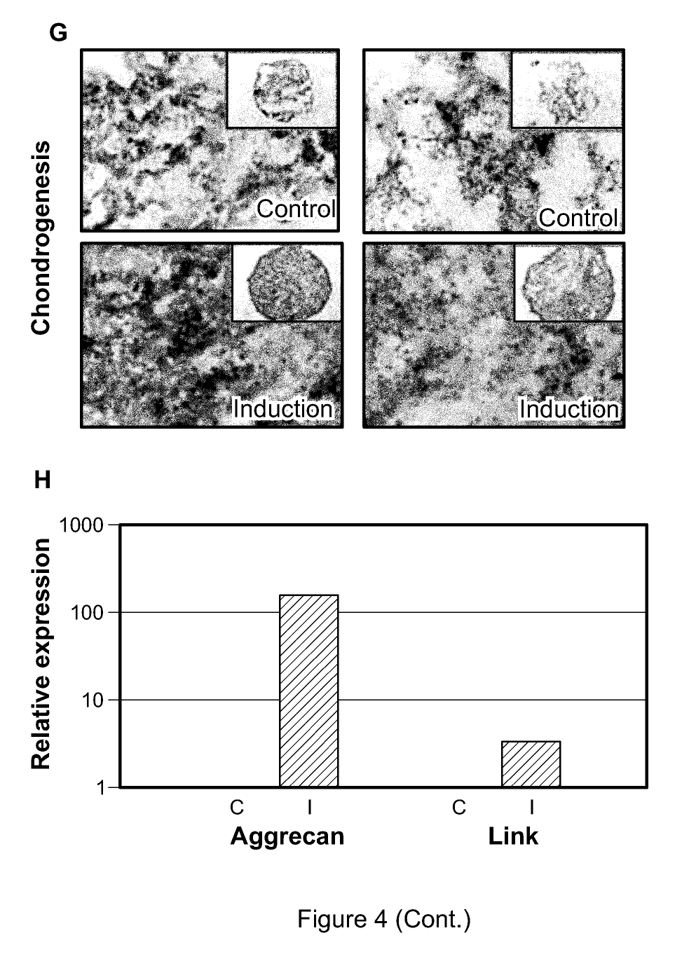

Specifically, GSCs and GSC-cs induced to adipogenic lineage displayed lipid vacuoles (FIG. 4A, C) as evidenced by increased oil red O accumulation in induced cells as compared to controls. Increased expression of lipoprotein lipase and PPARyIso2 (FIG. 4B) also was observed relative to non-induced controls. When subjected to osteogenic differentiation, GSCs and GSC-cs displayed calcium deposits typical of bone (FIG. 4D, F) and increased expression of osteocalcin and DLX5 (FIG. 4E) as compared to non-induced controls. The chondrogenic potential of GSCs and GSC-cs was confirmed by sulfated proteoglycans staining (FIG. 4G) and increased expression of aggrecan and link protein (FIG. 4H) as compared to non-induced controls after 28 days in culture. All together, these data clearly demonstrate that GSCs are easily differentiated into mesodermal lineage and have MSC properties.

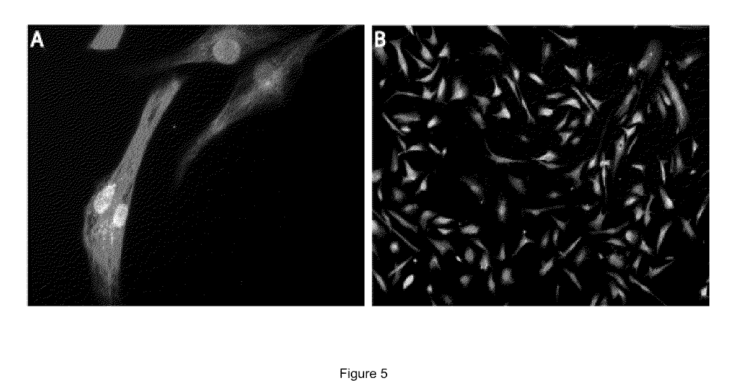

For cardiac differentiation, passage 2 cells were plated onto 12 well plates coated with human fibronectin or gelatin in DMEM high glucose with GlutaMax, supplemented as described in Example 1. After 24 hours, growth factors were removed and either 2 or 8 μM 5-AZA-2′-deoxycytidine (Aza) (Sigma Aldrich) was added. Media was changed every other day for 14 days. After 14 days, cells stained positive for the cardiac markers Demin and cardiac troponin T. See FIG. 5A.

### Example 4

### GSCs Differentiate into Cells of Ectodermal Lineage

For neural differentiation, passage 2 cells were plated onto 12 well dishes coated with matrigel (BD Pharmingen) or human fibronectin (Sigma Aldrich) and fed with KO DMEM+10% serum replacement+N-2 supplement (all from Invitrogen)+ITS premix (BD Biosciences)+glutamax (Invitrogen)+putrescine (Sigma Aldrich) with growth factors bFGF (20 ng/ml)+EGF (20 ng/ml) or growth factors SHH (200 ng/ml)+FGF8 (100 ng/ml)+bFGF (20 ng/ml) every other day for 12 days. Three (3) μm of retinoic acid (RA) was added to the cultures starting at day 10, daily for 3 days. Media was then aspirated and switched to DMEM-F/12 (Invitrogen)+10% serum replacement+N2 supplement+ITS premix+glutamax with growth factor EGF (20 ng/ml) or BDNF (20 ng/ml)+GDNF (20 ng/ml)+ascorbic acid (200 μm) for an additional 14 days. Cells were then stained on day 26 for the neural marker Nestin. Differentiated GSCs were positive for Nestin. See FIG. 5B.

### Other Embodiments

While the invention has been described in conjunction with the foregoing detailed description and examples, the foregoing description and examples are intended to illustrate and not to limit the scope of the invention, which is defined by the scope of the appended claims. Other aspects, advantages, and modifications are within the scope of the claims.

What is claimed is:

1. A purified population of adult human gonadal stem cells (GSCs), wherein said cells are positive for CD44, CD105, CD166, CD73, CD90, and STRO-1, negative for CD34, CD45, and HLA-DR, and do not express Vasa, Dazl, and Sox2.

2. The purified population of GSCs of claim 1, wherein said cells express vimentin, Oct4, and Nanog.

3. The purified population of GSCs of claim 1, wherein said cells are further positive for SSEA-4.

4. The purified population of GSCs of claim 1, wherein said cells are obtained from an adult testis sample.

5. The purified population of GSCs of claim 1, wherein said cells are capable of differentiating into cells of mesodermal lineage.

6. The purified population of GSCs of claim 5, wherein said cells are capable of differentiating into adipogenic cells, osteogenic cells, chondrogenic, and cardiogenic cells.

7. The purified population of GSCs of claim 1, wherein said cells have undergone at least 40 doublings in culture.

8. The purified population of GSCs of claim 1, wherein said cells have undergone at least 50 doublings in culture.

9. The purified population of GSCs of claim 1, wherein said cells have undergone at least 60 doublings in culture.

10. The purified population of GSCs of claim 1, wherein said cells comprise an exogenous nucleic acid.

11. The purified population of GSCs of claim 10, wherein said exogenous nucleic acid encodes a polypeptide.

12. The purified population of GSCs of claim 1, wherein said cells are housed within a scaffold.

13. The purified population of GSCs of claim 12, wherein said scaffold is biodegradable.

14. The purified population of GSCs of claim 13, wherein said biodegradable scaffold is composed of collagen.

15. The purified population of GSCs of claim 1, wherein said cells are capable of differentiating into cells of the ectodermal lineage.

16. The purified population of GSCs of claim 15, wherein said cells are capable of differentiating into neurogenic cells.

17. A clonal line of adult human GSCs, wherein said cells are positive for CD44, CD 105, CD166, CD73, and STRO-1, negative for CD34, CD45, CD90, and HLA-DR, and do not express Vasa, Dazl, and Sox2.

18. The clonal line of claim 17, wherein said wherein said cells are further positive for SSEA-4.

19. The clonal line of claim 17, wherein said cells are capable of differentiating into cells of mesodermal lineage.

20. The clonal line of claim 19, wherein said cells are capable of differentiating into adipogenic cells, osteogenic cells, and chondrogenic cells.

21. The clonal line of claim 17, wherein said cells comprise an exogenous nucleic acid.

22. The clonal line of claim 21, wherein said exogenous nucleic acid encodes a polypeptide.

23. The clonal line of claim 17, wherein said cells have undergone at least 40 doublings in culture.

24. The clonal line of claim 17, wherein said cells are housed within a scaffold.

25. The purified population of GSCs of claim 24, wherein said scaffold is biodegradable.

26. The purified population of GSCs of claim 25, wherein said biodegradable scaffold is composed of collagen.

27. A composition comprising the purified population of cells of claim 1 or the clonal line of claim 17 and a culture medium.

28. The composition of claim 27, wherein said composition further comprises a cryopreservative.

29. An article of manufacture comprising the purified population of cells of claim 1, or the clonal line of claim 17.

30. The article of manufacture of claim 29, wherein said purified population of cells or said clonal line is housed within a container.

31. The article of manufacture of claim 30, wherein said container is a vial or a bag.

32. The article of manufacture of claim 30, wherein said container further comprises a cryopreservative.

33. A method for purifying a population of GSCs from adult human testis, said method comprising obtaining cells from a human testis sample, culturing said human testis cells on a fibronectin coated substrate, and purifying said GSCs from said human testis cells by adherence to said fibronectin coated solid substrate, wherein said GSCs are positive for CD44, CD105, CD166, CD73, CD90, and STRO-1, negative for CD34, CD45, and HLA-DR, and do not express Vasa, Dazl, and Sox2.

34. The method of claim 33, wherein said cells express vimentin, Oct4, and Nanog.

35. The method of claim 33, wherein said cells are further positive for SSEA-4.

36. A method for culturing a population of GSCs from adult human testis, said method comprising obtaining a population of GSCs from adult human testis, wherein said GSCs are positive for CD44, CD105, CD166, CD73, CD90, and STRO-1, negative for CD34, CD45, and HLA-DR, and do not express Vasa, Dazl, and Sox2; and culturing said cells in the presence of a growth medium containing glucose, serum, fibroblast growth factor 2, and glial cell derived neurotrophic factor.
